(19) United States
(12) Patent Application Publication
(10) Pub. No.: US 2024/0066288 A1
(43) Pub. Date: Feb. 29, 2024

(54) Multipolar Cannula

(71) Applicant: Pajunk GmbH Medizintechnologie, Geisingen (DE)

(72) Inventors: Simone Pajunk-Schelling, Geisingen (DE); Martin Hauger, Donaueschingen (DE)

(73) Assignee: Pajunk GmbH Medizintechnologie, Geisingen (DE)

(21) Appl. No.: 18/494,612

(22) Filed: Oct. 25, 2023

Related U.S. Application Data
(62) Division of application No. 17/296,009, filed on May 21, 2021.

(30) Foreign Application Priority Data
Nov. 23, 2018 (DE) 10 2018 129 540.5

(51) Int. Cl. A61N 1/05; A61N 1/36; H01B 3/30

(52) U.S. Cl. CPC A61N 1/0502; A61N 1/3614; H01B 3/307

(57) ABSTRACT

A cannula with a cannula tube having a distal end and a proximal end and with a first electrode and at least one second electrode, wherein the cannula tube comprises a cannula tube body and a surface coating film electrically insulating the first and the second electrode with respect to one another, wherein the distal end of the cannula tube comprises a distal tip and at the proximal end an extension is disposed which comprises an electrically contacting connection for the electrodes, wherein the electrically insulating film and at least the second electrode is applied onto the cannula tube body using a thin film process.

### CROSS REFERENCE TO RELATED APPLICATIONS

This application is a divisional of U.S. patent application Ser. No. 17/296,009, filed May 21, 2021, which is a § 371 National Phase of PCT/EP20191081059, filed Nov. 12, 2019, which claims priority to German Patent Application No. 10 2018 129 540.5, filed Nov. 23, 2018.

### BACKGROUND

The present application relates to a multipolar cannula.

Known are multipolar cannulas, for example bipolar cannulas with a first electrode and a second electrode that are developed electrically insulated from one another. A known structure herein comprises applying onto cannula tube body an electrically insulating covering or coating in the form of a tube of an insulating synthetic material and sliding a further electrically conducting tube body onto the electrically insulating tube. A cannula developed in such manner thus has a large wall thickness and consequently a large cross section.

The present disclosure therefore addresses the problem of providing a multipolar cannula which can be of lesser dimension in order to decrease the risk of injury.

The application includes a multipolar cannula and a method for the production of a multipolar cannula having the features and structures recited herein.

The multipolar cannula according to the disclosure comprises a cannula tube having a distal end and a proximal end and a first electrode and at least one second electrode, wherein the cannula tube comprises a cannula tube body and a surface coating electrically insulating the first and the second electrode with respect to one another, wherein the distal end of the cannula tube comprises a distal tip, wherein the electrically insulating coating and at least the second electrode are applied onto the cannula tube body using a thin film process.

The application according to the disclosure of the electrically insulating coating or film and at least of the second electrode, using a thin film process, onto the cannula tube body enables obtaining significantly lesser cross sections of the cannula than the conventional structure of the multipolar cannula in the form of a double tube.

The first electrode is preferably formed by the cannula tube body whereby a compact structure is enabled.

According to an especially preferred embodiment, the electrically insulating film has a thickness of a few micrometers, preferably a thickness of less than 1 micrometer. The outer dimensions in the cross section of the multipolar cannula can thereby be significantly decreased.

The second electrode has preferably a thickness of a few micrometers, preferably a thickness of less than 1 micrometer. The diameter of the multipolar cannula can thereby be markedly decreased.

According to a preferred embodiment the electrically insulating film is comprised of parylene. Parylenes are suitable for surface coating onto the most diverse substrate materials and for surface coating the most diverse geometric objects such that they are especially suited for surface coating cannula tube bodies.

The electrically insulating film preferably covers a distal segment of the cannula tube body except for the distal tip or substantially completely. In this way good insulation between the cannula tube body and the second electrode applied in or on the insulating film can be enabled.

The second electrode is advantageously applied onto the electrically insulating film using a thin film process whereby a minimal layer thickness of the second electrode can be realized.

It is especially preferred for the second electrode to be comprised of aluminum since aluminum has good electric conductivity and, in addition, adheres well on different materials such as, for example, parylenes.

The second electrode is advantageously spaced apart from the distal end of the electrically insulating film and in particular covers the electrically insulating film except for a distal annularly circumferential segment. Due to the spacing of the electrically insulating film from the distal end good electrical insulation between the second electrode and the cannula tube body can be enabled. With the coverage of the electrically insulating film by the second electrode apart from a distal annularly circumferential segment, a second large-area electrode with good electrically conducting properties can be provided.

On the second electrode, at least in segments, a second electrically insulating film is advantageously disposed.

The second electrically insulating film is advantageously comprised of parylenes or white lacquer. In particular in the event the second electrode is fabricated of aluminum, it is advisable for the second electrically insulating film to be a white lacquer in order to cause the least possible impairment of the conductivity of the aluminum film.

The second electrically insulating film preferably covers the second electrode except for at least one distally disposed active segment to enable the safe handling of the cannula by a user.

It is feasible for each of the second electrodes to comprise more than one active segment whereby complex geometries of electrode structures can be enabled.

An especially preferred embodiment of the application provides for the second electrode to be disposed in the electrically insulating film. Such disposition can be attained thereby that the second electrode and the electrically insulating film are jointly applied onto the cannula tube body. This enables the embedding of the second electrode, or also of several second electrodes, in the electrically insulating film.

According to an especially preferred further development of the application, the first electrode and the second electrode are connectable to a bio-impedance sensor. Thereby a further functionality of the cannula is provided. On the one hand, the two electrodes within the frame of the multipolar cannula can be utilized for stimulation through appropriate stimulation loading.

If the two electrodes are connected to a bio-impedance sensor, it is possible to determine additionally in which type of tissue the tip of the multipolar cannula is disposed at any given time.

At the proximal end of the cannula tube an extension is preferably disposed which comprises an electrically contacting connection for the electrodes. Thereby the electrical contacting of the electrodes can be attained in simple manner, in particular if the electrodes extend over the entire length of the cannula tube from the distal end up to the electrically contacting connection.

The method according to the application for the production of a multipolar cannula with a cannula tube having a distal end and a proximal end and with a first electrode and at least one second electrode, wherein the cannula tube comprises a cannula tube body and a film electrically insulating the first and the second electrode with respect to one another, comprises the following steps:
Providing a cannula tube body and
Applying the electrically insulating film and the second electrode onto the cannula tube body using a thin film process.

By applying the electrically insulating film and the second electrode onto the cannula tube body using a thin film process the outer dimensions of the multipolar cannula, in particular the diameter, can be markedly decreased compared to conventional double-tube implementations of multipolar cannulas.

The electrically insulating film and at least one of the second electrodes are applied jointly in a thin film process and subsequently at least a distal segment of the at least one electrode is exposed by an ablation method, preferably by sputtering. In such a method complex geometries can be realized and in particular one or several second electrodes can be embedded in the electrically insulating film.

Alternatively, or additionally, for the application of the electrically insulating film and the at least one second electrode the following steps are executed.
Applying the electrically insulating film onto the cannula tube body and
Applying the second electrode onto the electrically insulating film.

Thereby a multilayer structure is obtained which offers technical advantages in manufacturing.

The electrically insulating film is preferably applied such that, except for the distal tip, the cannula tube body is substantially completely covered. Thereby good electrical insulation can be achieved.

The second electrode is advantageously applied onto the electrically insulating film such that the second electrode is disposed spaced apart from the distal end of the electrically insulating film and, in particular, the electrically insulating film is covered, except for a distal annularly circumferential segment. In this way the electrical insulation between the cannula tube body and the second electrically insulation film can be ensured.

After the second electrode has been applied, a second electrically insulating film can advantageously, at least in segments, be applied onto the second electrode in particular using a thin film process. The safety of usage, both, of the person handling the multipolar cannula as well as also that of the patient, can thereby be improved.

The second electrically insulating film is advantageously applied onto the second electrode such that the second electrode, except for at least one distally disposed active segment, is covered. Manifold feasibilities for implementing the geometry of the active segment of the second electrode are available. Onto the second electrically insulating film a third electrode and onto the third electrode a third electrically insulating film is preferably applied using a thin film process. Thereby a multilayered structure is obtained which simultaneously enables a multipole implementation of the cannula. It is understood that it is feasible to apply further electrodes in further films, wherein the outermost film should be implemented as electrically insulating film.

The thin film process is preferably a physical vapor deposition (PVD) process or a vapor deposition process or a sputter process or an imprinting method or a method for applying a lacquer film and/or a combination of several of said methods. Nearly any geometries of electrodes and electrically insulating coating films can be obtained using such methods.

### BRIEF DESCRIPTION OF DRAWINGS

The application will be explained in detail in conjunction with the following Drawing. Therein depict:

FIG. 1: a schematic perspective representation of a distal end of a first embodiment example of a multipolar cannula according to the application and

FIG. 2: a schematic perspective representation of a distal end of a second embodiment example of a multipolar cannula according to the application.

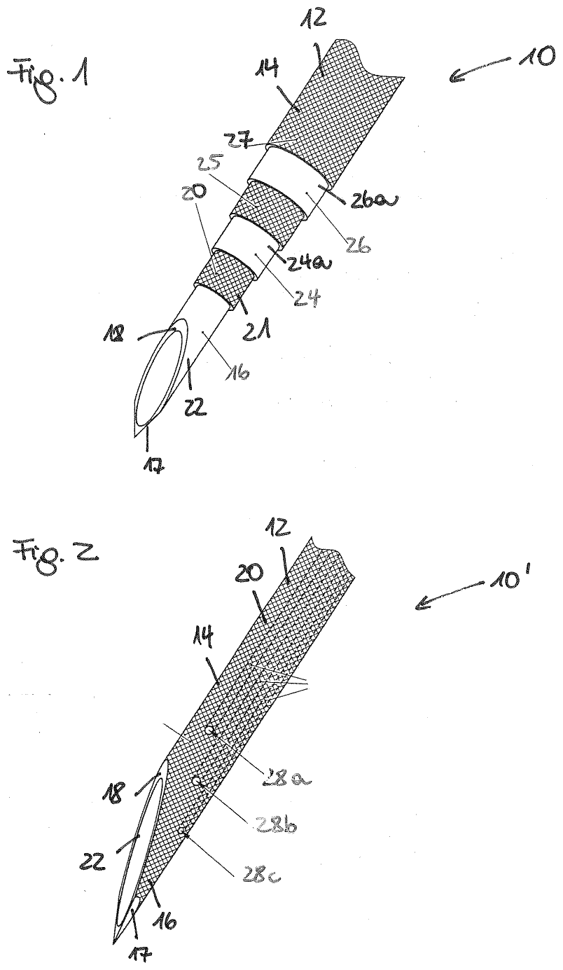

FIG. 1 shows a first embodiment example of a multipolar cannula 10 with a cannula tube 12 having a distal end 14 and a not depicted proximal end. The cannula tube 12 comprises a cannula tube body 18 and an electrically insulating film 20.

### DETAILED DESCRIPTION

The cannula tube body 18 comprises at the distal end 14 a distal tip 16 which can be formed, for example, thereby that the distal end 14 extends at an angle, for example at an angle of approximately 45°, obliquely with respect to the longitudinal axis of the cannula tube 12. The distal end of the distal tip 16 can additionally have a facet cut 17 to enhance the sharpness of the distal tip 16.

The electrically insulating film 20 is applied onto the cannula tube body 18 in a thin film process and covers, in particular circumferentially, the cannula tube body 18, wherein the distal tip 16 can remain exposed. The electrically insulating film 20 can be developed up to the proximal end of the cannula tube body 18. The cannula tube body 18 can herein form a first electrode 22.

On the electrically insulating film 20 a second electrode 24 is disposed which in particular is disposed circumferentially about the electrically insulating film 20 such that the distal end of the second electrode 24 is spaced apart from the distal end of the electrically insulating film 20 and, in particular, an annularly circumferential segment 21 of the electrically insulating film 20 remains exposed. Due to the circumferential segment 21, sufficient electrical insulation between the first electrode 22 and the second electrode 24 is also ensured at the active areas remaining exposed. The second electrode 24 can herein extend up to the proximal end of the cannula tube 18.

On the second electrode 24 is disposed a second electrically insulating film 25, in particular such that the second electrically insulating film 25 covers the second electrode 24 except for the at least one distally disposed active segment 24a. The active segment 24a can be developed, for example, such that it is annularly circumferential or it can assume nearly any geometric shape, in particular, it can be developed to be a circular, elliptical or rectangular surface.

If further poles for a multipolar cannula 10, as previously described, are desired, it is feasible, as is evident in the depicted embodiment example in FIG. 1, to apply onto the second electrically insulating film 25 a third electrode 26, preferably also using a thin film process, which, again, except for the at least one distally disposed active segment 26a, is covered by a third electrically insulating film 27.

The cannula 10 can be supplemented with further poles in this manner.

At the proximal end of the multipolar cannula 10 electrodes 22, 24, 26 can be contacted such that they are electrically conducting, such that across the electrodes 22, 24, 26 electrical stimulation is feasible when introducing the multipolar cannula 10 into the body of a patient.

To be able to provide further functionalities, there is also the feasibility of connecting the first electrode 22 and the second electrode 24 to a bio-impedance sensor.

The multipolar cannula 10 according to the embodiment depicted in FIG. 1 can be manufactured in the following manner. First, the cannula tube body 18 is provided. Subsequently, the electrically insulating film 20 is applied onto the cannula tube body 18 in a thin film process, in particular such that the distal end 14 of the cannula tube body 18 remains exposed, providing a first electrode 22. Onto the electrically insulating film 20 subsequently the second electrode 24 is applied using a thin film process, in particular such that the electrically insulating film 20, except for the annularly circumferential segment 21, which adjoins the distal end of the electrically insulating film 20, is covered. Onto the second electrode 24 subsequently a second electrically insulating film 25 is applied using a thin film process, in particular such that an annularly circumferential active segment 24a of the second electrode 24 remains exposed.

If further poles on the multipolar cannula 10 are desired and, consequently, an expansion to a multipolar cannula is intended, onto the second electrically insulating film 25 a third electrode 26, in particular using a thin film process, can optionally be applied, in particular such that the second electrically insulating film 25, except for an annularly circumferential segment, is covered. Onto the third electrode 26 a third electrically insulation film 27 can subsequently be applied, in particular such that the third electrode 26, except for an active segment 26a which is in particular developed annularly circumferentially, is covered.

FIG. 2 shows a further embodiment example of a multipolar cannula 10′ which, like the multipolar cannula 10 according to the first embodiment example, comprises the cannula tube 12 having a distal end 14 and a, not depicted, proximal end, which comprises a cannula tube body 18 and an electrically insulating film 20. The cannula tube body 18 represents again the first electrode 22.

The multipolar cannula 10′ according to the second embodiment example differs from the first embodiment example in that in the electrically insulating film 20 at least one, in the present embodiment example three, second electrodes 28a, 28b, 28c are embedded. The electrodes 28a, 28b, 28c, are developed as track conductors in the electrically insulating film 20 and extend from the distal region of the cannula tube 20 up to the proximal end. They can reach up to the distal tip 16 of the cannula tube 20. The active regions of the electrodes 28a, 28b, 28c, can be exposed by removing the electrically insulating film 20 over the distal ends of electrodes 28a, 28b, 28c. In the embodiment example the electrodes 28a, 28b, 28c are developed as essentially round track conductors extending parallel to one another. However, it is also evident that the electrodes can assume manifold geometric physical forms.

A further difference between the second embodiment example of the multipolar cannula 10′ and the first embodiment example 10 is that the electrically insulating film 20 covers the entire cannula tube body 18 up over the distal tip 16 and only exposes the front face of the cannula tube body 18 as well as optionally provided facet cut faces 17.

The multipolar cannula 10′ is manufactured in the following manner:

First, the cannula tube body 18 is provided. Subsequently, in a thin film process the electrically insulating film 20 as well as the electrodes 28a, 28b, 28c, embedded in the electrically insulating film 20, are applied jointly, wherein the electrodes 28a, 28b, 28c can be, for example, imprinted and subsequently a distal segment of electrodes 28a, 28b, 28c is exposed by using an ablation process, for example sputtering, in order to form the particular active segments of the corresponding electrodes 28a, 28b, 28c.

### LIST OF REFERENCE NUMBERS

10 Multipolar cannula
10′ Multipolar cannula
12 Cannula tube
14 Distal end
16 Distal tip
17 Facet cut
18 Cannula tube body
20 Electrically insulating film
21 Segment
22 First electrode
24 Second electrode
24a Active segment
25 Second electrically insulating film
26 Third electrode
26a Active segment
27 Third electrically insulating film
28a Electrode
28b Electrode
28c Electrode

1. A method for the production of a multipolar cannula with a cannula tube having a distal end and a proximal end and with a first electrode and at least one second electrode, wherein the cannula tube comprises a cannula tube body and a film electrically insulating the first and the second electrode with respect to one another, comprising the steps:
providing a cannula tube body,
applying the electrically insulating film and the second electrode onto the cannula tube body using a thin film process.

2. The method as in claim 1, wherein the electrically insulating film and at least one of the electrodes are applied jointly in a thin film process and subsequently a distal segment of the electrode is exposed using an ablation process, preferably sputtering.

3. The method as in claim 1, wherein for the applying of the electrically insulating film and of the second electrode the following steps are executed:
applying the electrically insulating film onto the cannula tube body,
applying the second electrode onto the electrically insulating film.

4. The method as in claim 3, wherein the electrically insulating film is applied such that the cannula tube body is completely covered or completely covered except for the distal tip.

5. The method as in claim 3, wherein the second electrode is applied onto the electrically insulating film such that the second electrode is disposed spaced apart from the distal end of the electrically insulating film and the electrically insulating film is covered except for a distal annularly circumferential segment.

6. The method as in claim 3, wherein after the application of the second electrode a second electrically insulating film is applied at least in segments onto the second electrode using a thin film process.

7. The method as in claim 6, wherein the second electrically insulating film is applied onto the second electrode such that the second electrode is covered except for at least one distally disposed active segment.

8. The method as in claim 6, further comprising
applying a third electrode onto the second electrically insulating film, and
applying a third electrically insulating film onto the third electrode using a thin film process.

9. The method as in claim 1, wherein the thin film process is a PVD process, a vapor deposition process, or a sputter process or an imprinting process, a method for applying a lacquer film, or a combination thereof.
